(19) United States
(12) Patent Application Publication
(10) Pub. No.: US 2025/0009981 A1
(43) Pub. Date: Jan. 9, 2025

(54) A subassembly of a medicament delivery device

(71) Applicant: SHL Medical AG, Zug (CH)

(72) Inventor: Meng-Jhen Chiou, Taoyuan City (TW)

(21) Appl. No.: 18/711,947

(22) PCT Filed: Nov. 18, 2022
(86) PCT No.: PCT/EP2022/082361

§ 371 Date: May 21, 2024

(30) Foreign Application Priority Data
Dec. 2, 2021 (EP) 21211951.5

(51) Int. Cl. A61M 5/32; A61M 5/20

(52) U.S. Cl. CPC A61M 5/3232; A61M 5/2033

(57) ABSTRACT

The present disclosure generally relates to medicament delivery devices such as autoinjectors, and particularly concerns a subassembly adapted to prevent injuries to a user caused by a medicament delivery member at a proximal end of the medicament delivery device.

### TECHNICAL FIELD

The present disclosure generally relates to medicament delivery devices such as autoinjectors, and particularly concerns a subassembly adapted to prevent injuries to a user caused by a medicament delivery member at a proximal end of the medicament delivery device.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning preventing injuries to a user caused by a needle of the medicament delivery devices, which are set out in more detail below.

### SUMMARY

An object of the present disclosure is to provide a subassembly for a medicament delivery device which solves, or at least mitigates problems of the prior art.

According to a first aspect of the present disclosure, there is provided a subassembly of a medicament delivery device for expelling medicament from a medicament container. The subassembly comprises a housing having a proximal end and a distal end. A cover structure is configured to surround a medicament delivery member at a proximal end of the medicament delivery device, the cover structure is movable inside the housing in an axial direction of the housing between a retracted position in which the medicament delivery member is exposed at the proximal end of the cover structure, and an extended position in which the medicament delivery member is covered by the cover structure. The cover structure comprises a slot in a surface facing radially outwards towards the housing. The housing comprises a slot in a surface facing radially inwards towards the cover structure. The subassembly further comprising a guiding member arranged between the cover structure and the housing in the slot of the housing and in the slot of the cover structure. The slot of the housing and the slot of the cover structure are adapted so that, when the cover structure is moved towards the retracted position, the guiding member slides in the slot of the cover structure, and along a first path in the slot of the housing from a first position to a second position. Further, when the cover structure is moved from the retracted position towards the extended position, the guiding member slides in the slot of the cover structure, and along a second path in the slot of the housing from the second position to a locking position. The slot of the housing and the slot of the cover structure are adapted so that, once in the locking position, if the cover structure is again attempted to be moved towards the retracted position, the guiding member maintains the relative positions of the slot of the housing and the slot of cover structure thereby preventing the cover structure from moving axially to the retracted position.

Embodiments of the present disclosure advantageously provides for a lockout function for the cover structure so that once a user has used the medicament delivery device, thus having pushed the cover structure to the retracted position, and subsequently released to the extended position, the guiding member has been guided by the slots to a locking position. In the locking position, the guiding member acts as an interlock to ensure that the cover structure cannot move in relation to the housing back to the retracted position. In other words, when the cover structure is for the first time moved to the retracted position and subsequently to the extended position, the guiding member travels along the slots and reaches the locking position which it cannot escape from during an attempt to again move the cover structure towards the retracted position. In this way, after use, the cover structure is maintained in the extended position such that a user is prevented from sharp injury caused by a medicament delivery member, such as a needle.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

The same direction terminology has been used to describe other components such as the pad—for example, the proximal end of the pad is the part of the pad closest to the dose delivery site (injection site), and the distal end of the pad is the part of the pad furthest from the dose delivery site. In the Figures, the longitudinal direction is the direction of axis 102, with the corresponding circumferential direction 31 and radial direction 32 relative to the axis 102 also shown.

When the wording ‘at the injection site’ or ‘at the dose delivery site’ is used in this application, it generally refers to the point where the medicament delivery device (e.g., a needle) enters the patient, along with the surrounding area, for example the area where the pad is attached.

According to one embodiment, the guiding member may be a separate part adapted to fit in the slots. In other words, the guiding member is not attached or anchored to any other part of the subassembly or the medicament delivery device. The guiding member thus lies loose in the slots and its motion is determined by the shape of the slots.

According to one embodiment, the guiding member may be held in place in the slots by the proximity of the housing and the cover structure. In other words, the guiding member may be enclosed by the slots although a small gap between the cover structure and the housing is allowed, as long as the guiding member cannot slip through the gap. Thus, the gap needs to be smaller than a thickness, or smallest dimension, of the guiding member.

According to one embodiment, the guiding member may comprise a circular cross-section.

According to one embodiment, the guiding member may be disc-shaped. A disc-shaped guiding member allows for relatively low friction between side walls of the slots and the guiding members, thereby improving the reliability of the motion of the guiding member in the slots.

According to one embodiment, the guiding member may be moved in the slot of the housing by a force exerted on the guiding member from side walls of the slot in the cover structure. In other words, the side walls of the cover structure may push the guiding member along the slot in the housing.

According to one embodiment, the slot of the cover structure may be arranged to extend along a circumference of the cover structure. Thus, the slot of the cover structure may extend along a curved path substantially following an outer circumference of the cover structure which may be cylindrical. The slot may be curved around an axis parallel with the axial direction of the medicament delivery device.

According to one embodiment, a path along the main extension of the slot of the cover structure may be perpendicular to an axis parallel with the axial direction of the medicament delivery device.

According to one embodiment, the first path of the slot of the housing may comprise a first portion and a second portion, where the second portion is inclined with respect to the first portion and arranged to extend at least partly along a circumference of a surface of the housing, so that when the guiding member slides in the second portion it simultaneously moves in the slot of the cover structure in a first direction. The inclined second portion is inclined also with respect to the slot of the cover structure. The slot of the cover structure forces the guiding member to move along the inclined second portion, while a counterforce from the inclined second portion forces the guiding member to move along the slot of the cover structure.

According to one embodiment, the first portion of the slot of the housing may be arranged substantially parallel with a longitudinal axis of the housing.

According to one embodiment, the guiding member may be moved in the slot in the cover structure by a force exerted on the guiding member from side walls of the second portion of the slot in the housing.

According to one embodiment, the second path of the housing slot may comprise a shoulder wall portion configured to prevent the guiding member from moving from the lock position towards the second position. The shoulder wall portion may be substantially parallel with the wall portions of the slot in the cover structure.

According to one embodiment, the second path of the slot of the housing may comprise a main portion and a locking portion connected by an intermediate portion, the intermediate portion may be configured so that when the guiding member moves in the intermediate portion the guiding member is simultaneously caused to move along the slot in the cover structure until the guiding member reaches the locking portion which includes the locking position.

According to one embodiment, the main portion may extend parallel with the axial direction of the medicament delivery device, the intermediate portion may be inclined with respect to the main portion, and the locking portion may be parallel with the main portion. Further, the main extension of the slot of the cover structure may be perpendicular to the main longitudinal extension of the main portion of the slot of the housing.

According to one embodiment, when the guiding member moves in the slot of the housing from the first position to the second position the guiding member may move in the slot of the cover structure in a first direction, and when the guiding member moves in the slot of the housing from the second position to the locking position the guiding member may move in the slot of the cover structure in a second direction being opposite to the first direction.

According to one embodiment, in the locking position, the guiding member may abut on the shoulder wall portion and a corresponding side wall of the slot in the cover structure being parallel with the shoulder wall portion.

There is further provided a medicament delivery device comprising the subassembly of any of the herein disclosed embodiments.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2 illustrates a subassembly according to embodiments of the present disclosure;

FIG. 3 is a cross-section of the housing in FIG. 2 that shows a slot in the inner surface of the housing;

FIG. 4 illustrates a guiding member according to embodiments of the present disclosure;

FIG. 5 illustrates a partial view from the proximal end of the subassembly illustrating the guiding member in place between the housing and the cover structure;

FIG. 6 illustrates the cover structure in the extended position with the guiding member in an initial first position;

FIG. 7 illustrates the cover structure in the retracted position with the needle exposed, and the guiding member in a second position;

FIG. 8 illustrates the cover structure in the extended position with the guiding member in a locking position; and

FIG. 9 illustrates the cover structure in the extended position with the guiding member in a locking position with the guiding member abutting against a side wall.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

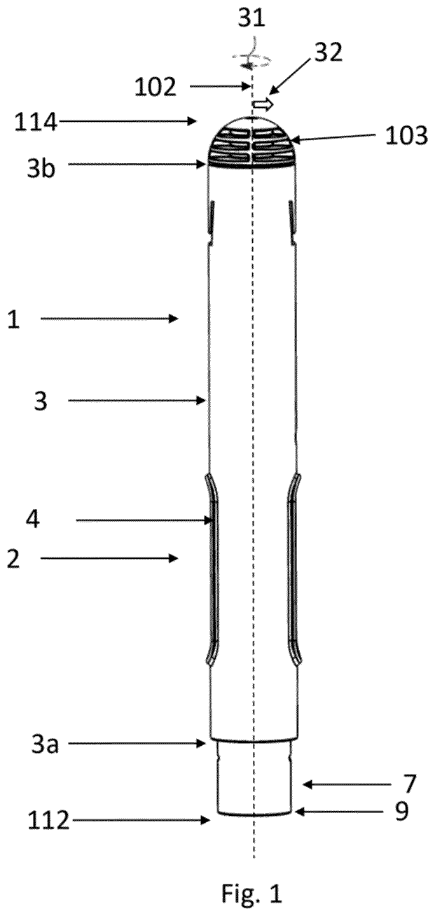

FIG. 1 shows an example of a medicament delivery device 1 such as an autoinjector according to embodiments of the present disclosure. The medicament delivery device 1 is configured to expel medicament from a medicament container via a medicament delivery device, better seen in FIG. 7, to a user at a dose delivery site. The medicament delivery device 1 extends from a proximal end 112 to a distal end 114 relative to the axis 102.

The medicament delivery device 1 comprises a housing 3 with a window 4. The housing 3 has a proximal end 3a and a distal end 3b. A rear cap 103 covers the distal end 3b of the housing 3.

The medicament delivery device 1 comprises a cover structure 7. The cover structure 7 is arranged in the housing 3 and extends proximally from the proximal end 3a. The housing 3 and the cover structure 7 are part of a subassembly 2.

The cover structure 7 is configured to be moved linearly relative to the housing 3 from an extended position shown in FIG. 1 to a retracted position in which the cover structure 7 is received further in the housing 3 and in which a medicament delivery member such as a needle is exposed.

The cover structure 7 is biased in the proximal direction towards the extended position.

The medicament delivery device 1 comprises the subassembly 2 which will now be described in more detail with reference to subsequent drawings.

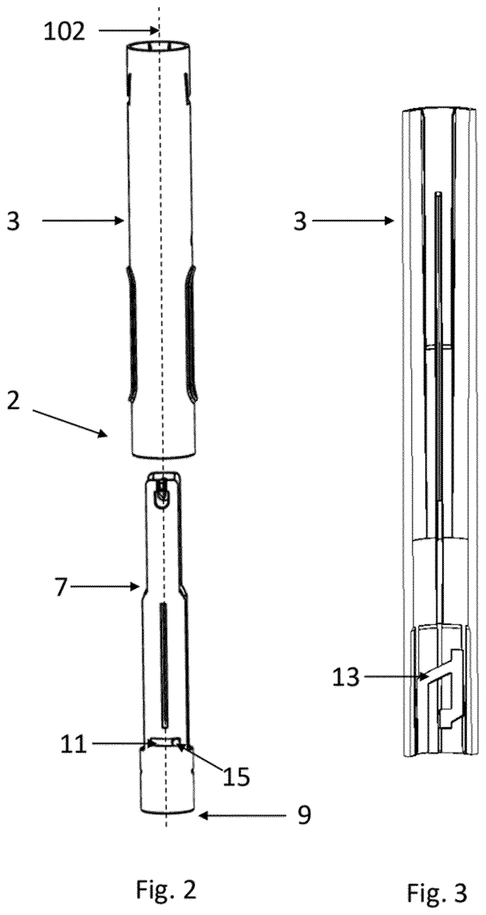

FIG. 2 illustrates a subassembly 2 according to embodiments of the present disclosure. The subassembly comprises the housing 3 and the cover structure 7 which has been axially displaced in the drawing to better illustrate different parts of the subassembly 2.

As described above, the cover structure 7 is configured to surround a medicament delivery member at a proximal end 112 of the medicament delivery device. The cover structure 7 is movable inside the housing 3 in an axial direction of the housing 3 between a retracted position in which the medicament delivery member is exposed at the proximal end 9 of the cover structure 7, and an extended position in which the medicament delivery member is covered by the cover structure 7.

Further, the cover structure 7 comprises a slot 11 in a surface facing radially outwards towards the housing 3.

Similarly, the housing 3 comprises a slot 13 in a surface facing radially inwards towards the cover structure 7. The slot 13 is shown in the cross-section view in FIG. 3, showing the inner side of the housing 3 that faces the slot 11 of the cover structure 7.

The subassembly 2 further comprises a guiding member 15 arranged between the cover structure 7 and the housing 3 in the slot 13 of the housing and in the slot 11 of the cover structure 7. In other words, the slots 11 and 13 are oriented relative each other so that the guiding member 15 can be arranged in both slots 11 and 13 simultaneously. It can be understood that the slots 11 and 13 overlap at the location of the guiding member 15.

Preferably, the guiding member 15 is a separate part adapted to fit in the slots 11 and 13. Thus a width of the guiding member 15 is smaller than a width of the slots 11 and 13. The slots 11 and 13 are generally elongated with the length of the slots being of larger dimension than the widths thereof. The motion of the cover structure 7 relative to the housing 3 causes the guiding member to slide in the slots 11 and 13. If the housing 3 and cover structure 7 are disassembled, the guiding member 15 falls out.

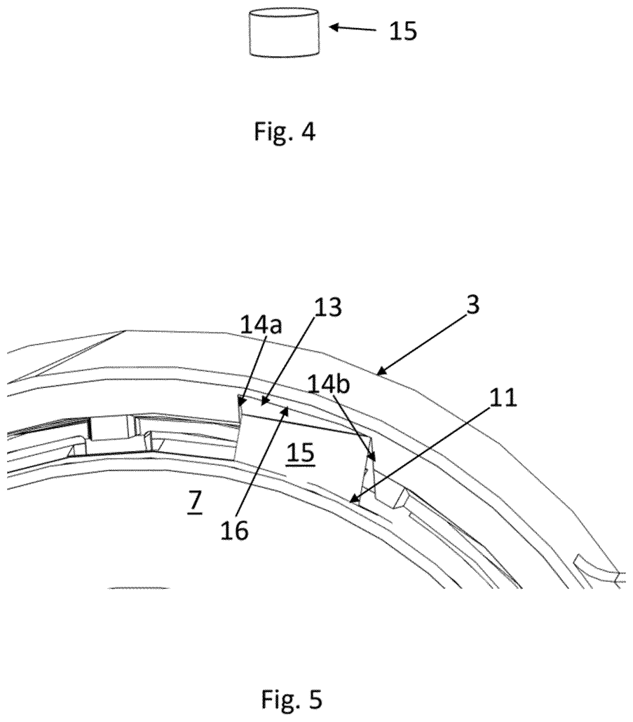

FIG. 4 illustrates an example guiding member 15 according to preferred embodiments of the present disclosure. The guiding member 15 comprises a circular cross-section and is in FIG. 4 provided in a disc-shape. However, other shapes such as spheres or elliptical shapes are also conceivable.

FIG. 5 illustrates a partial view from the proximal end 3a of the housing 3 where the guiding member 15 is shown in place in the slot 13 of the housing and in the slot 11 of the cover structure 7. The guiding member 15 is arranged between the housing 3 and the cover structure 7 and is held in place in the slots 11 and 13 by the proximity of the housing 3 and the cover structure 7. The spacing between the housing 3 and cover structure 7 is in other words sufficiently small so that the guiding member 15 cannot fall out from the slots 11 and 13.

The slot 13 has a bottom 16 having a curved shape that substantially follows a circumference of the housing 3. In other words, the slot 13 is curved. In a similar way the slot 11 is also curved. The side walls 14a and 14b that connects with the bottom 16 are in this example embodiment parallel. The angles between the plane of the bottom 16 and the respective side walls are not orthogonal due to the curved shape of the slot 13. The slot 11 has a similar structure, e.g., a curved bottom surface and parallel side walls that may not be orthogonal to the bottom surface.

When the cover structure 7, often called a needle guard, has moved once from the extended position to the retracted position thereby exposing the needle so that a medicament can be ejected, the medicament delivery device has been used and should be disposed of. After use, the cover structure 7 is again moved to the extended position by a biasing force provided by for example a spring to cover the needle. However, it is desirable to make sure that the cover structure cannot again be moved to the retracted position such that the needle can cause injury. This is one problem addressed by embodiments of the present disclosure.

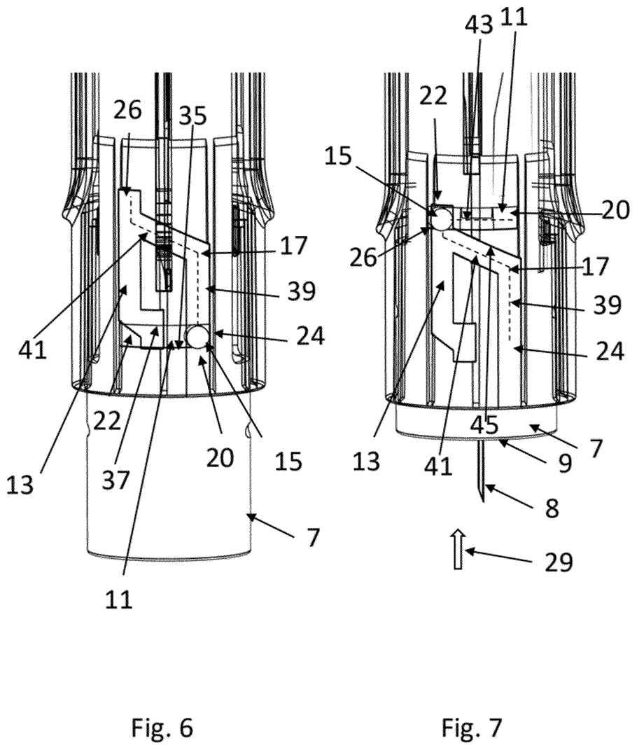

More specifically, the slot 13 of the housing and the slot 11 of the cover structure are specifically adapted so that the guiding member 15 travels in the slots 11 and 13 to a final locking position, after the cover structure 7 is moved to the extended position after the medicament delivery device has been used, in which final locking position the guiding member 15 locks the cover structure 7 in the extended position. This will now be described in more detail with reference to FIGS. 6-9.

FIG. 6 illustrates the cover structure in the extended position covering the needle 8. The guiding member 15 is located at an initial position on one end of the slot 11 of the cover structure 7. FIG. 7 illustrates the cover structure 7 in the retracted position with the needle 8 exposed at the proximal end 9 of the cover structure 7. The slot 13 of the housing 3 and the slot 11 of the cover structure 7 are adapted so that, with the guiding member 15 in the initial position at one end 20 of the slot 11, and when the cover structure 7 is moved from the extended position shown in FIG. 6, towards the retracted position shown in FIG. 7, the guiding member 15 slides in the slot 11 of the cover structure towards the other end 22, and along a first path 17 in the slot 13 of the housing 3 from a first position 24 to a second position 26.

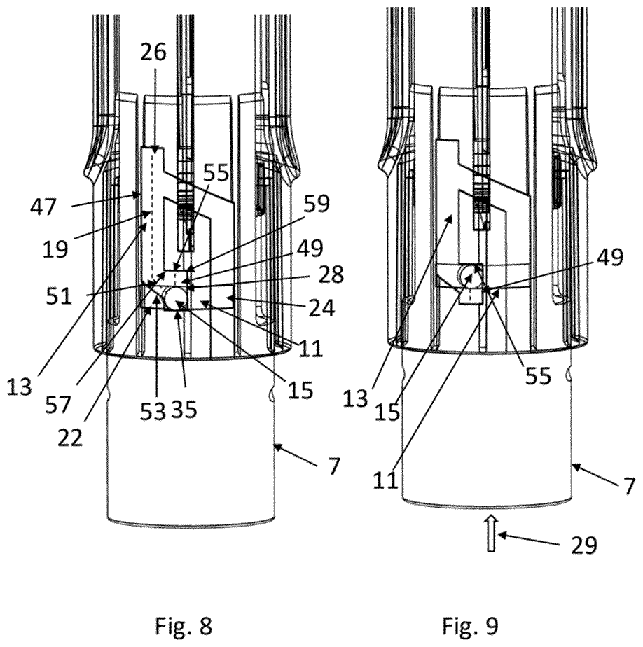

Turning now to FIG. 8 in which the cover structure has subsequently been moved from the retracted position towards the extended position. During this motion, the guiding member 15 slides in the slot 11 of the cover structure 7, and along a second path 19 in the slot of the housing 3 from the second position 26 to a locking position 28.

The slot 13 of the housing 3 and the slot 11 of the cover structure 7 are adapted so that, once in the locking position 28, if the cover structure 7 is again attempted to be moved towards the retracted position as illustrated in FIG. 9, the guiding member 15 maintains the relative positions of the slot 13 of the housing 3 and the slot 11 of cover structure 7 thereby preventing the cover structure 7 from moving axially to the retracted position. In other words, if the cover structure 7 is moved in the axial direction indicated by arrow 29, the guiding member 15 being moved by the motion of the slot 11 is prevented from travelling in a reverse direction in the slot 13 to the second position 26 after having reached the locking position 28. Note that that the locking position 28 may allow a small movement of the cover structure 7, thus, to maintain the relative positions of the slot 13 of the housing 3 and the slot 11 of cover structure 7 is interpreted as preventing the cover structure from moving too far so that the needle 8 is exposed.

The guiding member 15 is an independent part that is not anchored to any other part of the medicament delivery member 1. The guiding member 15 is free to travel along the slots 11 and 13 although the motion of the guiding member 15 is determined by the motion of the cover structure 7. Turning again to FIGS. 6 and 7, when the cover structure 7 is moved in the axial direction 29 towards the retracted position, the slot 11 pushes on the guiding member 15 so that the guiding member 15 travels distally along the first path 17 in the slot 13. Thus, the guiding member 15 is moved in the slot 13 of the housing by a force exerted on the guiding member 15 from side walls of the slot 11 in the cover structure 7. When moving towards the retracted position, the proximal side wall 35 pushes on the guiding member 15. When moving from the retracted position with the guiding member 15 in the second position 26, the distal side wall 37 pushes on the guiding member 15 so that it moves proximally along the second path 19.

The first path 17 of the slot 13 of the housing 3 comprises a first portion 39 and a second portion 41. The second portion 41 is inclined with respect to the first portion 39 and arranged to extend at least partly along a circumference of a surface of the housing 3. In this way, when the guiding member 15 slides in the second portion 41 it simultaneously moves in the slot 11 of the cover structure 7 in a first direction along path 43. Since the path 17 in the first portion 39 is substantially perpendicular to the path 43 of the slot 11, the guiding member 15 will only move along the first portion 39 and not in the slot 11 before the guiding member 15 reaches the second portion 41. The path 17 in the second portion 41 is at least partly arranged along the circumference of the housing 3 and thus not perpendicular to the path 43 of the slot 11, meaning that the second portion 41 of the slot 13 will force the guiding member 15 to travel sideways along the slot 11 until the end of the second portion 41, where the guiding member enters the second path 19 of the slot 13. The motion of the guiding member 15 in the slot 11 is possible as the path 43 of the slot 11 of the cover structure 7 is arranged to extend along a circumference of the cover structure 7 thereby allowing the guiding member to travel circumferentially along the curved path 43. In the second portion 41 a force is exerted on the guiding member 15 from a distal side wall 45 of the second portion 41 to cause the motion of the guiding member 15 in the slot 11 of the cover structure 7.

The first portion 39 of the slot of the housing is arranged substantially parallel with a longitudinal axis 102 of the housing. The main extension of the slot 11 along path 43 is perpendicular to an axis parallel with the longitudinal axis 102.

Since the cover structure is generally cylindrical, the slot 11 is curved whereby the guiding member 15 travels along the curved path 43 provided by the curved slot 11 around part of the circumference of the cover structure 7.

When the guiding member 15 moves in the slot 13 of the housing 3 from the first position 24 to the second position 26, the guiding member 15 moves in the slot 11 of the cover structure 7 in a first direction.

Now with reference to FIGS. 8 and 9, when the guiding member 15 moves in the slot 13 of the housing 3 from the second position 26 to the locking position 28, the guiding member 15 moves in the slot 11 of the cover structure 7 in a second direction being opposite to the first direction. Thus, the shape of the slot 13 is such that the guiding member 15 is caused to move in opposite directions during a full cycle.

To achieve such opposite motion of the guiding member 15 in the slot 11, the second path 19 of the housing slot 13 comprises a main portion 47 and a locking portion 49 connected by an intermediate portion 51. The intermediate portion 51 is configured so that when the guiding member 15 moves in the intermediate portion 51 the guiding member 15 is simultaneously caused to move along the path 43 of the slot 11 in the cover structure 7 until the guiding member 15 reaches the locking portion 49 which includes the locking position 28.

The main portion 47 extends parallel with the longitudinal axis 102 of the medicament delivery device 1. The main portion 47 of the second path 19 of the slot 13 is parallel with the first portion 39 of the first path 17. The intermediate portion 51 of the second path 19 is inclined with respect to the main portion 47 and the locking portion 49 is parallel with the main portion 47. Similar to the inclined second portion 41 causing a motion of the guiding member 15 along the path 43 of the slot 11, the inclined intermediate portion 51 causes a motion of the guiding member 15 along the path 43 in the opposite direction, i.e., by a force exerted by the proximal side wall 53 of the intermediate portion 51 forcing the guiding member 15 to move along the path 43.

The guiding member 15 reaches the locking portion 49 which is parallel with the main portion 47, and perpendicular with the path 43 of the slot 11. Thus, if the cover structure 7 is again moved towards the retracted position, as shown in FIG. 9, there is no inclined surface or sidewall that will cause the guiding member 15 to move along the path 43, whereby the guiding member maintains in the locking portion 49.

A shoulder wall portion 55 of the locking portion 49 is configured to prevent the guiding member 15 from moving from the lock position 28 towards the second position 26. In other words, the shoulder wall portion 55 is shaped or designed so that when the guiding member 15 is pushed against the shoulder wall portion 55, the guiding member 15 does not slide reversely in the intermediate portion 51 out from the locking portion 49, thus the guiding member 15 is maintained in the locking portion 49.

The shoulder portion 55 may be designed in various ways to prevent the guiding member 15 from escaping from the locking portion 49. For example, an inclination of the shoulder wall portion 55 with respect to the proximal side wall 35 of the slot 11 may be such that the guiding member 15 does not slide towards the intermediate portion 51 and out from the locking portion 49. In one possible implementation, the point 57, indicating the corner at one end of the shoulder portion 55 at the outlet of the locking portion 49, may be more proximal than a point 59 of the shoulder wall portion further from the end at point 57 of the shoulder portion 55, here at the other corner at the opposite end of the shoulder wall portion 55. However, the point 59 may be at an intermediate position of the shoulder wall portion 55 which thereby forms a triangular shape, or pocket for the guiding member 15.

Further, in other possible implementations, the proximal side wall 35 of the slot 11 is inclined such that the end of the proximal side wall 35 at the position 22 is more distal than the other end at the position 24, or more distal than an intermediate point of the side wall portion 35 that for example coincides with the corner at point 59 of the shoulder wall portion 55, so that the guiding member 15 does not slide along path 43 and out from the locking portion 49 by interaction with the shoulder wall portion 55.

In other possible implementations, the shoulder wall portion 55 may be at least partly parallel with the proximal side wall 35 of the slot 11 so that the guiding member 15 does not move along the path 43 in the slot 11, whereby, as the guiding member 15 abuts against the shoulder wall portion 55 and the corresponding side wall 35, the guiding member locks the cover structure so that it cannot be moved to the retracted position.

The slot 13 comprises a first portion 39 which follows a straight path parallel with the longitudinal axis 102. The first portion 39 connects with a second portion 41 which is inclined with the first portion 39 and follows a curved path provided by the circumference of the housing 3. The second portion 41 also extends partly along the longitudinal axis 102. The angle between the direction of a path of the first portion 39 and the direction of a path of the second portion 41 is below 90 degrees, such as in the range of about 30-55 degrees. The second portion 41 connects with the main portion 47 of the second path 19. The main portion 47 of the second path 19 is parallel with the first portion 39 of the first path 17. Further, the main portion 47 of the second path 19 and the first portion 39 of the first path 17 are arranged next to each other, e.g., they extend so that the guiding member travels firstly towards the distal end 3b of the housing 3 in the first portion, and subsequently towards the proximal end 3a in the main portion 47 of the second path 19.

The main portion 47 connects with the intermediate portion which is inclined with the main portion. The angle between the direction of a path of the main portion 47 and the direction of a path of the intermediate portion 51 is below 90 degrees, such as in the range of about 30-55 degrees. The intermediate portion 51 connects with the locking portion 49, the path of which is parallel with the path of the main portion 47.

Further, the slots 11 and 13 are curved according to the curved shape of the cover structure 7 and the housing 3, respectively.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the support pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-16. (canceled)

17. A subassembly of a medicament delivery device for expelling medicament from a medicament container, the subassembly comprising:
a housing having a proximal end and a distal end; and
a cover structure configured to surround a medicament delivery member at a proximal end of the medicament delivery device, the cover structure is movable inside the housing in an axial direction of the housing between a retracted position in which the medicament delivery member is exposed at the proximal end of the cover structure, and an extended position in which the medicament delivery member is covered by the cover structure,
the cover structure comprising a slot in a surface facing radially outwards towards the housing,
the housing comprising a slot in a surface facing radially inwards towards the cover structure,
the subassembly further comprising a guiding member arranged between the cover structure and the housing in the slot of the housing and in the slot of the cover structure,
wherein the slot of the housing and the slot of the cover structure are adapted so that, when the cover structure is moved towards the retracted position, the guiding member slides in the slot of the cover structure, and along a first path in the slot of the housing from a first position to a second position, and
when the cover structure is moved from the retracted position towards the extended position, the guiding member slides in the slot of the cover structure, and along a second path in the slot of the housing from the second position to a locking position,
the slot of the housing and the slot of the cover structure are adapted so that, once in the locking position, if the cover structure is again attempted to be moved towards the retracted position, the guiding member maintains the relative positions of the slot of the housing and the slot of the cover structure thereby preventing the cover structure from moving axially to the retracted position.

18. The subassembly according to claim 17, wherein the guiding member is a separate part adapted to fit in the slot of the cover structure and in the slot of the housing.

19. The subassembly according to claim 17, wherein the guiding member is held in place in the slot of the cover structure and in the slot of the housing by the proximity of the housing and the cover structure.

20. The subassembly according to claim 17, wherein the guiding member comprises a circular cross-section.

21. The subassembly according to claim 17, wherein the guiding member is moved in the slot of the housing by a force exerted on the guiding member from side walls of the slot in the cover structure.

22. The subassembly according to claim 17, wherein the slot of the cover structure is arranged to extend along a circumference of the cover structure.

23. The subassembly according to claim 17, wherein the first path of the slot of the housing comprises a first portion and a second portion, where the second portion is inclined with respect to the first portion and arranged to extend at least partly along a circumference of a surface of the housing, so that when the guiding member slides in the second portion it simultaneously moves in the slot of the cover structure in a first direction.

24. The subassembly according to claim 23, wherein the first portion of the slot of the housing is arranged substantially parallel with a longitudinal axis of the housing.

25. The subassembly according to claim 23, wherein the guiding member is moved in the slot in the cover structure by a force exerted on the guiding member from side walls of the slot in the housing in the second portion.

26. The subassembly according to claim 17, wherein, the second path of the slot of the housing comprises a shoulder wall portion configured to prevent the guiding member from moving from the lock position towards the second position.

27. The subassembly according to claim 17, wherein the second path of the slot of the housing comprises a main portion and a locking portion connected by an intermediate portion, the intermediate portion configured so that when the guiding member moves in the intermediate portion the guiding member is simultaneously caused to move along the slot in the cover structure until the guiding member reaches the locking portion which includes the locking position.

28. The subassembly according to claim 27, wherein main portion extends parallel with axial direction of the medicament delivery device, the intermediate portion is inclined with respect to the main portion, and the locking portion is parallel with the main portion.

29. The subassembly according to claim 17, wherein the slots are configured so that when the guiding member moves in the slot of the housing from the first position to the second position the guiding member moves in the slot of the cover structure in a first direction, and when the guiding member moves in the slot of the housing from the second position to the locking position the guiding member moves in the slot of the cover structure in a second direction being opposite to the first direction.

30. The subassembly according to claim 27, wherein, in the locking position, the guiding member abuts the shoulder wall portion and a corresponding side wall of the slot in the cover structure being parallel with the shoulder wall portion.

31. The subassembly according to claim 17, wherein the cover structure is biased towards the extended position, for example by a spring.

32. A medicament delivery device comprising the subassembly according to claim 17.
